(19) United States
(12) Patent Application Publication
(10) Pub. No.: US 2022/0248806 A1
(43) Pub. Date: Aug. 11, 2022

(54) FOOTWEAR INSERT ASSEMBLY

(71) Applicant: FRANKIE4 IP 1 Pty Ltd, Indooroopilly, QLD (AU)

(72) Inventor: Caroline Rose Haire, Indooroopilly (AU)

(73) Assignee: FRANKIE4 IP 1 Pty Ltd, Indooroopilly, QLD (AU)

(21) Appl. No.: 17/631,085

(22) PCT Filed: Jul. 31, 2020
(86) PCT No.: PCT/AU2020/050789

§ 371 Date: Jan. 28, 2022

(30) Foreign Application Priority Data
Aug. 1, 2019 (AU) 2019902759

(51) Int. Cl. A43B 17/16; A43B 7/144; A43B 17/02

(52) U.S. Cl. CPC A43B 17/16; A43B 17/023; A43B 7/144

(57) ABSTRACT

A footwear insert assembly including a pair of integrally formed full length footbeds adapted for insertion within an item of footwear and a heel insert adapted for insertion in the item of footwear beneath one of the full length footbeds of the pair of integrally formed full length footbeds such that the full length footbed and the heel insert cooperate with the footwear to raise a heel portion of the full length footbed. The pair of full length footbeds include a first contoured footbed having a contoured arch portion, the contoured arch portion of the first contoured footbed having a thickness providing a first degree of arch support, and a second contoured footbed comprising a second contoured footbed having a contoured arch portion, the contoured arch portion of the second contoured footbed having a thickness that is less than the thickness of the contoured arch portion of the first contoured footbed, the contoured arch portion of the second contoured footbed providing a second degree of arch support, wherein each of the first contoured footbed and the second contoured footbed is adapted for interchangeable insertion within an item of footwear to thereby produce a supportive feel when worn.

### FIELD

The present disclosure relates to a footwear insert assembly for providing footwear with at least one removable and interchangeable footbed. In particular, the disclosure relates to a footwear insert assembly for shoes to alleviate, treat or reduce foot and lower limb pain using insertable footbeds and inserts.

### BACKGROUND

Reference to background art herein is not to be construed as an admission that such art constitutes common general knowledge.

During running and gait, a wearer's shoe contacts the ground such that the foot lands on a lateral side of the heel first before the centre of the load moves to the medial side of the foot. During this process, the foot naturally pronates. However, in many people, the wearer's foot and ankle overpronate (i.e. the ankle rolls too far downward and inward with each step and continues to roll when the foot should be starting to push off). Overpronation is particularly common in people with so-called “flat feet” but can occur in anyone. This overpronation can lead to injuries, particularly overuse type injuries, due to increased and repetitive stress on the muscles, tendons and ligaments of the foot and lower leg includes the shin and knees as the limb rotates too far inwardly.

In addition, many people also suffer from knee, hip and back pain which can be caused by differences in the lengths of their respective legs but may also be symptomatic of another issue that is exacerbated by excessive load on the Achilles tendon.

Providing shoes that correct or prevent overpronation that cater to different types of feet and wearer preferences while also being comfortable to wear for extended periods is difficult.

### SUMMARY OF THE INVENTION

In one form, although it need not be the only or indeed the broadest form, there is provided a footwear insert assembly comprising:

a footbed adapted for insertion within an item of footwear; and

a heel insert adapted for insertion beneath the footbed such that the footbed and the heel insert cooperate with the footwear to raise a heel portion of the footbed.

In another form, there is provided a footwear insert assembly comprising:

a full length footbed adapted for insertion within an item of footwear; and

a heel insert adapted for insertion beneath the full length footbed such that the full length footbed and the heel insert cooperate with the footwear to raise a heel portion of the full length footbed.

In another form, there is provided a method for providing a range of fittings and/or supports to an item of footwear, the method comprising the steps of:

providing a first contoured full length footbed having a first degree of arch support and a heel insert, wherein the first contoured full length footbed is integrally formed;

providing a second contoured full length footbed having a second degree of arch support, wherein the second contoured full length footbed is integrally formed;

inserting the heel insert into an item of footwear at a heel portion of the item of footwear; and

inserting the first contoured full length footbed into the item of footwear, wherein the heel insert is located between a heel portion of the first contoured full length footbed and a heel portion of the item of footwear;

removing the first contoured full length footbed from within the item of footwear; and

inserting the second contoured full length footbed into the item of footwear, wherein the heel insert is located between a heel portion of the second contoured full length footbed and a heel portion of the item of footwear.

In another form, there is provided a footwear insert assembly comprising:

a container;

a pair of items of footwear located in the container;

two pairs of integrally formed full length footbeds adapted for insertion within an item of footwear of the pair of items of footwear, each pair of integrally formed full length footbeds comprising:
a first contoured footbed having a contoured arch portion, the contoured arch portion of the first contoured footbed having a thickness providing a first degree of arch support;
a second contoured footbed comprising a second contoured footbed having a contoured arch portion, the contoured arch portion of the second contoured footbed having a thickness that is less than the thickness of the contoured arch portion of the first contoured footbed, the contoured arch portion of the second contoured footbed providing a second degree of arch support; and

a pair of heel inserts, each heel insert adapted for insertion beneath one footbed of each of the pairs of footbeds such that the footbed and the heel insert cooperate within the footwear to raise a heel portion of the footbed.

In yet another form, there is provided a footwear insert assembly comprising:

a pair of integrally formed full length footbeds adapted for insertion within an item of footwear, the pair of full length footbeds comprising:
a first contoured footbed having a contoured arch portion, the contoured arch portion of the first contoured footbed having a thickness providing a first degree of arch support;
a second contoured footbed comprising a second contoured footbed having a contoured arch portion, the contoured arch portion of the second contoured footbed having a thickness that is less than the thickness of the contoured arch portion of the first contoured footbed, the contoured arch portion of the second contoured footbed providing a second degree of arch support;
wherein each of the first contoured footbed and the second contoured footbed is adapted for interchangeable insertion within an item of footwear to thereby produce a supportive feel when worn; and

a heel insert adapted for insertion in the item of footwear beneath one of the full length footbeds of the pair of integrally formed full length footbeds such that the full length footbed and the heel insert cooperate with the footwear to raise a heel portion of the full length footbed.

Preferably, the footwear insert assembly further comprises at least one of:

a half length footbed adapted for insertion within the item of footwear to provide a wide fitting; and

a forefoot insert adapted for insertion beneath the full length footbed within the footwear so that the full length footbed and the forefoot insert cooperate with the footwear to produce a narrow fitting.

Preferably, in use, the full length footbed having the heel insert beneath a heel portion of the full length footbed is inserted into the item of footwear to provide a raised heel portion of the full length footbed.

Preferably, in use, the full length footbed having the forefoot insert beneath a forward portion of the footbed is inserted into the item of footwear to provide a narrow fit.

Preferably, in use, the full length footbed having the heel insert beneath a heel portion of the full length footbed is inserted into the item of footwear to provide a raised heel portion of the full length footbed, and the forefoot insert beneath a forward portion of the footbed is inserted into the item of footwear to provide a narrow fit.

Preferably, the heel insert is also adapted for insertion beneath the half length footbed. Preferably, in use, the half length footbed having the heel insert beneath a heel portion of the half length footbed is inserted into the item of footwear to provide a raised heel portion of half length footbed and a wide fit.

Preferably, the footbed comprises a first contoured footbed having a contoured arch portion, the contoured arch portion of the first contoured footbed having a thickness providing a first degree of arch support. Preferably, the first contoured footbed comprises a first full length footbed. More preferably, the assembly further comprises a second contoured footbed having a contoured arch portion, the contoured arch portion of the second contoured footbed having a thickness that is less than the thickness of the contoured arch portion of the first contoured footbed, the contoured arch portion of the second contoured footbed providing a second degree of arch support. Preferably, the second contoured footbed comprises a second full length footbed. Preferably, each of the first contoured footbed and the second contoured footbed is adapted for insertion within an item of footwear to thereby produce a supportive feel when worn. Preferably, each of the first contoured footbed and the second contoured footbed comprises a first contoured full length footbed and a second contoured full length footbed, respectively. Alternatively, each of the first contoured footbed and the second contoured footbed comprises a first contoured half length footbed and a second contoured half length footbed, respectively. Further alternatively, one of the first and second contoured footbeds comprises a contoured full length footbed and the other of the first and second contoured footbeds comprises a contoured half length footbed.

Preferably, the footwear insert assembly comprises a pair of footbeds, each footbed adapted for insertion within an item of footwear, such as a pair of shoes, for example.

Preferably, the full length footbed and the half length footbed are interchangeably and/or removably insertable into an item of footwear. Suitably, each of the full length footbeds and the half length footbed are removably insertable within an item of footwear.

Preferably, each of the first and second contoured footbeds comprises a medial side and a lateral side. Preferably, the contoured arch portion of the first and/or second contoured footbed is located along the medial side. Preferably, the contoured arch portion is a cushioned or contoured portion adapted to support an arch of a foot in the item of footwear to reduce or prevent excessive pronation.

Preferably, a footwear insert assembly as described above may further comprise one or more items of footwear. Suitably, the assembly may comprise a shoe or pair of shoes adapted for use with the footbed/s and heel insert/s.

Preferably, the heel insert comprises a heel raising insert adapted to raise and/or elevate the heel. Alternatively, the heel insert comprises a heel wedge insert adapted to elevate the heel. Further preferably, the heel wedge insert is adapted to raise and tilt the heel in a varus or valgus position (e.g. outward or inward manner, respectively). In some embodiments, the heel wedge insert comprises a varus wedge insert or a valgus wedge insert.

Preferably, a length of the heel raising insert is approximately half of a length of each of the full length footbeds. Preferably, the length of the heel raising insert extends approximately halfway along the length of the full length footbed.

Preferably, the heel raising insert comprises a first end and a second end. Preferably, the first end has a first height and the second end has a second height. Preferably, the second height is less than the first height. Preferably, the gradient of a surface of the heel raising insert between the first end and the second end is less than 0.08, and more preferably less than 0.05. In some preferable embodiments, the gradient of the surface of the heel raising insert is between approximately 0.02 and 0.08, and, even more preferably, the gradient of the surface of the heel raising insert is between approximately 0.03 and 0.05.

In another form, there is provided a footwear insert assembly comprising:

a container;

a pair of shoes located in the container; and

a pair of footbeds, each footbed adapted for insertion within one shoe of the pair of shoes; and

a pair of heel inserts, each heel insert adapted for insertion beneath one footbed of the pair of footbeds such that the footbed and the heel insert cooperate within the footwear to raise a heel portion of the footbed.

Preferably, the container comprises a shoebox.

In another form, there is provided a method for providing a range of fittings and/or supports to a shoe, the method comprising the steps of:

providing a first contoured full length footbed having a first degree of arch support and a heel insert;

inserting the heel insert into an item of footwear at a heel portion of the item of footwear; and

inserting the first contoured full length footbed into the item of footwear, wherein the heel insert is located between a heel portion of the first contoured full length footbed and a heel portion of the item of footwear.

Preferably, the method further comprises the steps of:

providing a second contoured full length footbed having a second degree of arch support;

removing the first contoured full length footbed from within the item of footwear; and

inserting the second contoured full length footbed into the item of footwear, wherein the heel insert is located between a heel portion of the second contoured full length footbed and a heel portion of the item of footwear.

In another form, there is provided a footwear insert assembly comprising:

a first contoured footbed having a contoured arch portion, the contoured arch portion of the first contoured footbed having a thickness providing a first degree of arch support; and

a second contoured footbed having a contoured arch portion, the contoured arch portion of the second contoured footbed having a thickness that is less than the thickness of the contoured arch portion of the first contoured footbed, the contoured arch portion of the second contoured footbed providing a second degree of arch support,

wherein each of the first contoured footbed and the second contoured footbed is adapted for insertion within an item of footwear to thereby produce a supportive feel when worn.

In yet another form, there is provided a footwear insert assembly comprising a heel insert.

Preferably, the heel insert comprises a heel raising insert adapted to raise and/or elevate the heel. Alternatively, the heel insert comprises a heel wedge insert adapted to elevate the heel. Further preferably, the heel wedge insert is adapted to raise and tilt the heel in a varus or valgus position (e.g. outward or inward manner, respectively). In some embodiments, the heel wedge insert comprises a varus wedge insert or a valgus wedge insert.

Preferably, a length of the heel raising insert is approximately half of a length of each of the full length footbeds. Preferably, the length of the heel raising insert extends approximately halfway along the length of the full length footbed.

Preferably, the heel raising insert comprises a first end and a second end. Preferably, the first end has a first height and the second end has a second height. Preferably, the second height is less than the first height. Preferably, the gradient of a surface of the heel raising insert between the first end and the second end is less than 0.08, and more preferably less than 0.05. In some preferable embodiments, the gradient of the surface of the heel raising insert is between approximately 0.02 and 0.08, and, even more preferably, the gradient of the surface of the heel raising insert is between approximately 0.03 and 0.05.

Preferably, the full length footbed is integrally formed or formed as a single piece.

### BRIEF DESCRIPTION OF THE DRAWINGS

By way of example only, preferred embodiments of the invention will be described more fully hereinafter with reference to the accompanying figures, wherein:

FIG. 1 illustrates a footwear insert assembly having a contoured full length footbed and a heel raising insert;

FIG. 2 illustrates a forefoot insert that may be provided with the footwear insert assembly of FIG. 1;

FIG. 3 illustrates an overhead view of the contoured full length footbed of FIG. 1;

FIG. 4 illustrates a side view of the contoured full length footbed of FIGS. 1 and 3;

FIG. 5 illustrates a lateral cross-section of the contoured full length footbed of FIG. 1;

FIG. 6 illustrates the contoured full length footbed inserted into a shoe;

FIG. 7 illustrates a cross-section of the shoe having the contoured full length footbed inserted therein;

FIG. 8 illustrates a side view of the contoured full length footbed with the heel raising insert placed underneath;

FIG. 9 illustrates another side view of the contoured full length footbed with the heel raising insert placed underneath;

FIG. 10 illustrates a cross-section of a shoe having the contoured full length footbed and the heel raising insert inserted therein;

FIG. 11 illustrates a top view of the full length contoured footbed with the forefoot insert placed underneath;

FIG. 12 illustrates a cross-section of a shoe having the contoured full length footbed with the heel raising insert and forefoot inserted arranged underneath;

FIG. 13 illustrates a side view of a second contoured full length footbed according to another embodiment;

FIG. 14 illustrates a lateral cross-section of the second contoured full length footbed;

FIG. 15 illustrates a top view of a contoured half length footbed;

FIG. 16 illustrates a perspective view of the contoured half length footbed;

FIG. 17 illustrates a side view of the contoured half length footbed;

FIG. 18 illustrates a lateral cross-section of the contoured half length footbed;

FIG. 19 illustrates a container having a pair of shoes, a pair of contoured full length footbeds and a pair of heel raising inserts;

FIG. 20 illustrates a side view of a heel insert in the form of a heel wedge;

FIGS. 21-22 illustrate an exemplary heel raising insert in accordance with an embodiment of the present invention; and

FIGS. 23-24 illustrate the heel raising insert shown in FIGS. 21-22 in use with the contoured full length footbed.

Preferred features, embodiments and variations of the invention can be discerned from the following Detailed Description which provides sufficient information for those skilled in the art to perform embodiments of the invention. The Detailed Description and associated figures are not to be regarded as limiting the scope of the preceding Summary of the Invention in any way.

### DETAILED DESCRIPTION OF THE PREFERRED EMBODIMENTS

The present invention relates to a footwear insert assembly having an integrally formed footbed for insertion within a shoe and a heel insert for insertion between the shoe and the footbed.

More particularly, the invention relates to a footwear insert assembly comprising a contoured full length footbed designed to reduce or prevent excessive pronation and a heel insert positionable under the contoured full length footbed to lift part of the full length footbed in the footwear to reduce or alleviate foot and/or lower limb pain and/or address leg length discrepancies in a wearer.

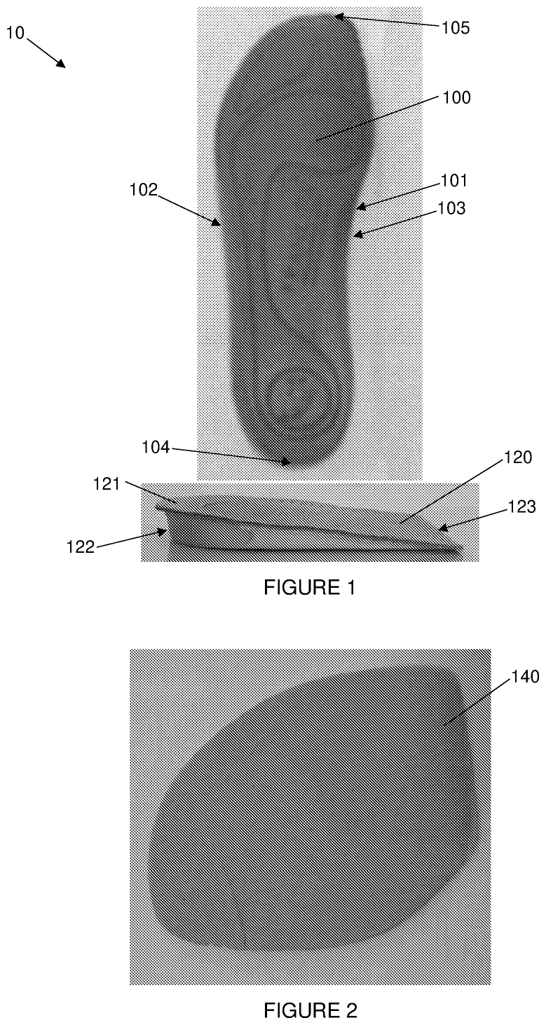

FIG. 1 illustrates a footwear insert assembly 10 having two components: a footbed in the form of a contoured full length footbed 100 and a heel insert in the form of a heel raising insert 120. The heel raising insert 120, having a uniform height or thickness along a lateral cross-section of the heel insert, is complementary to the shape of the contoured full length footbed 100 such that the combination of the heel raising insert 120 and contoured full length footbed 100 provide a substantially even lift of the heel and support beneath the heel of a user. More particularly, the heel raising insert 120 uniformly tapers from a thickest end, which is located under the heel of a user when in use, to a thinnest end, which is located under a middle area of the foot of a user when in use.

It should be appreciated that some irregularity may be experienced in the lift of the heel in use due to the differing shapes and distributions of different user's heels.

The footwear insert assembly 10 is designed or intended to be used with an item of footwear, such as a shoe. It should be appreciated that the footwear insert assembly 10, and other embodiments of footwear insert assemblies described herein, can be utilised with any type of shoe (including dress shoes, casual shoes and sport shoes, for example) and can be utilised with men's, women's and kid's shoes, as required.

The contoured full length footbed 100 is inserted into the shoe, either alone or in combination with the heel raising insert 120, which is located between the contoured full length footbed 100 and the midsole or sole of the shoe (an example of this can be seen in FIG. 10). A useful feature of the footwear insert assembly 10 is that the contoured full length footbed 100 can be removed and interchanged with other types of footbeds. Additionally, the heel raising insert 120 can, advantageously, be inserted and removed from beneath the contoured full length footbed 100, as desired or required.

A forefoot insert 140 is shown in FIG. 2. In some embodiments, the forefoot insert 140 may be provided with the footwear insert assembly 10. The forefoot insert 140 will described in more detail below.

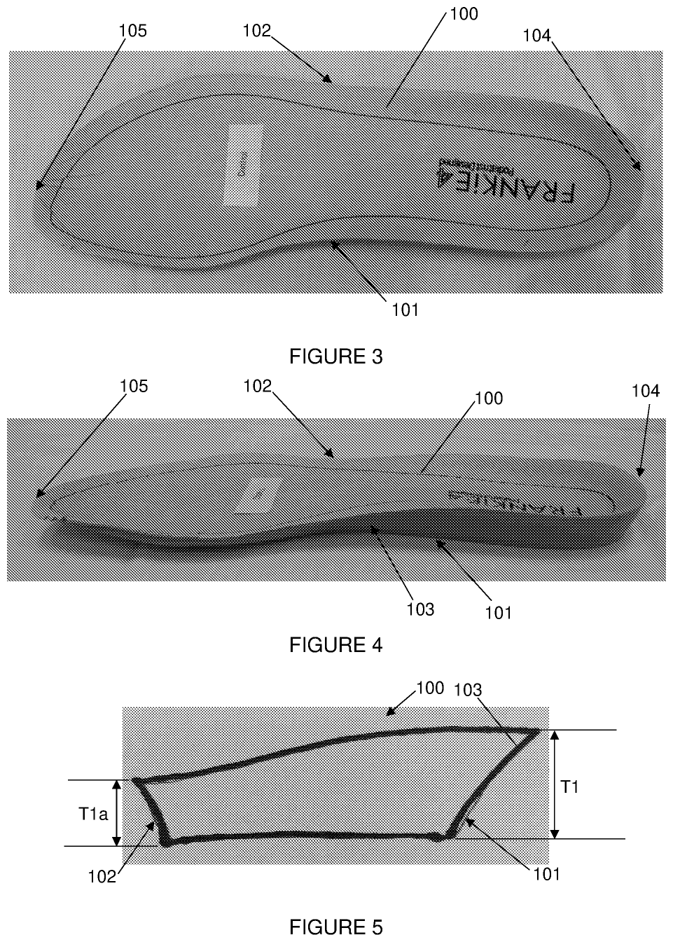

The contoured full length footbed 100 (also shown in FIGS. 3-6) is an integrally formed (i.e. formed as a single piece or article) “control” or “supportive” footbed and includes a contoured medial side 101 (opposite a lateral side 102) and is designed for a user who has a pronating foot tending toward overpronation or “flat footedness”. A contoured arch portion 103 having thickness T1 providing a first degree of arch support (see FIG. 5) is located on the medial side 101 of the footbed 100 and, in use, supports the arch and reduces or prevents the foot from excessively pronating to mitigate stress and/or injuries. The opposing lateral side 102 has a thickness T1a which is less than thickness T1 of the medial side 101.

The contoured full length footbed 100 also includes a heel portion 104 opposite a toe portion 105.

The heel raising insert 120 is substantially complementary to the shape of the heel portion 104 of the contoured full length footbed 100 such that the combination of the heel raising insert 120 and contoured full length footbed 100 provide a substantially even lift of the heel and support beneath the heel of a user. More particularly, the heel raising insert 120 and the heel portion 104 of the contoured full length footbed 100 have the same width.

The heel raising insert 120 can be made from any suitable material that will not completely deform under the weight of a foot when the wearer's weight is applied to it. However, it should be appreciated that the heel raising insert 120 may be made from a soft material to provide cushioning and shock absorption but may also be made from a firmer, denser and/or more resilient material to better accommodate leg length differences or to provide an increase in height for a wearer who wishes to appear taller.

The forefoot insert 140 is substantially complementary to the shape of the toe portion 105 of the contoured full length footbed 100 such that the combination of the forefoot insert 140 and contoured full length footbed 100 provide a lift to the forefoot and support beneath the forefoot of a user. More particularly, the forefoot insert 140 and the toe portion 105 of the contoured full length footbed 100 have the same width.

With particular reference to FIGS. 3 and 4, the contoured arch portion 103 on the medial side 101 reduces and/or prevents the flattening of the arch of the foot which occurs during overpronation.

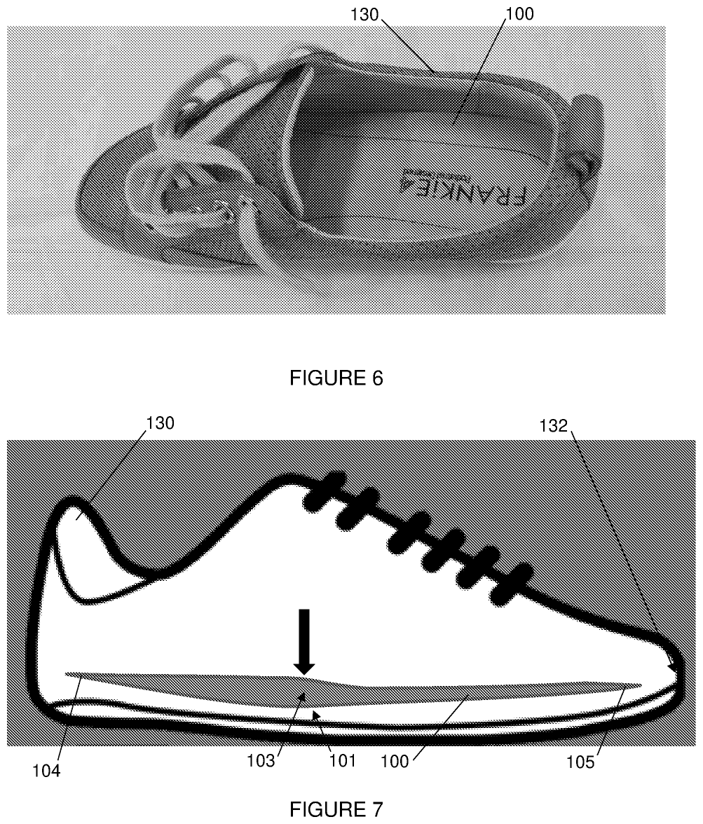

In the embodiment illustrated in FIGS. 6 and 7, as a shoe 130, into which the contoured full length footbed 100 has been inserted, worn by a person strikes the ground or a surface, the arch of the foot of the person wearing the shoe 130 exerts downward force (as indicated by the downward arrow in FIG. 7) on the contoured arch portion 103 on the medial side 101. As the contoured arch portion 103 is made of a sufficiently resilient material, such as foam or rubber, the contoured arch portion 103 does not fully or completely flatten out, thereby supporting the arch and preventing overpronation.

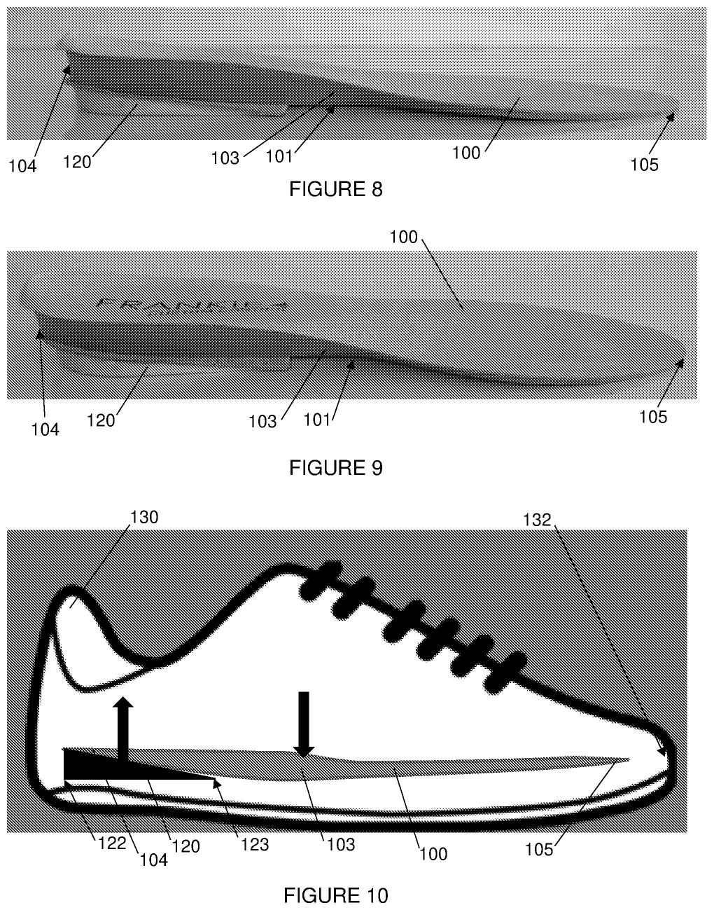

Turning to FIGS. 8-10, there is shown the heel raising insert 120 located beneath the heel portion 104 of the contoured full length footbed 100. The heel raising insert 120 elevates or raises the heel portion 104 of the contoured full length footbed 100 which has been shown to release pressure on the arch of the foot and posterior leg. This is particularly useful for treating foot and lower limb pain, or leg length discrepancies (i.e. one leg shorter than the other) which are known to cause significant pain to sufferers of such a condition.

The heel raising insert 120, as seen in FIG. 1 and in cross-section in FIG. 10, has a substantially wedge-shaped design. As a result of the wedge shaped, the heel raising insert 120 has a sloped, declining surface 121 extending from a top of rear portion 122 of the heel raising insert 120 toward a forward portion 123 of the heel raising insert 120. When inserted into the shoe 130, the rear portion 122 is proximate the rear 134 of the shoe 130.

The heel raising insert 120 can be attached to an underside of the contoured full length footbed 100 by any suitable means, including using an adhesive as an example.

When used in conjunction with contoured full length footbed 100, as the shoe 130 worn by a person strikes the ground or a surface, the arch of the foot of the person wearing the shoe 130 exerts downward force on the contoured arch portion 103 on the medial side 101 (as indicated by the downward arrow over contoured arch portion 103 in FIG. 10), as described above. As the contoured arch portion 103 is made of a sufficiently resilient material, such as foam or rubber, the contoured arch portion 103 does not fully or completely flatten out, thereby supporting the arch and preventing overpronation. The presence of the heel raising insert 120, which is also sufficiently resilient, maintains the heel portion 104 of the contoured full length footbed 100, and thus the heel of the wearer, at an elevated position, relative to the remainder of the contoured full length footbed 100, thereby reducing load on the Achilles tendon and other associated ligaments and tendons. In some embodiments where the heel raising insert 120 is used to address leg length discrepancies, the heel raising insert 120 ensures the shorter leg is compensated for by the additional height provided by the heel raising insert 120. It has also been found that the use of a heel insert in combination with a contoured footbed may also provide improved treatment of plantar heel pain compared to the use of a heel insert alone. It should be appreciated that plantar heel pain is commonly termed “plantar fasciitis” or ‘plantar heel pain syndrome’ and posterior heel pain may be related to Achilles problems or bursitis.

As foot pain is very commonly located in the back half of the foot, these conditions may be addressed, and the symptoms of those conditions alleviated, by the heel raising insert which reduces load on the posterior of the foot shifting it in a more anterior, or forward, direction. Effectively, weight is shifted from the heel or back of the foot, which is prone to pain, to the front of the foot.

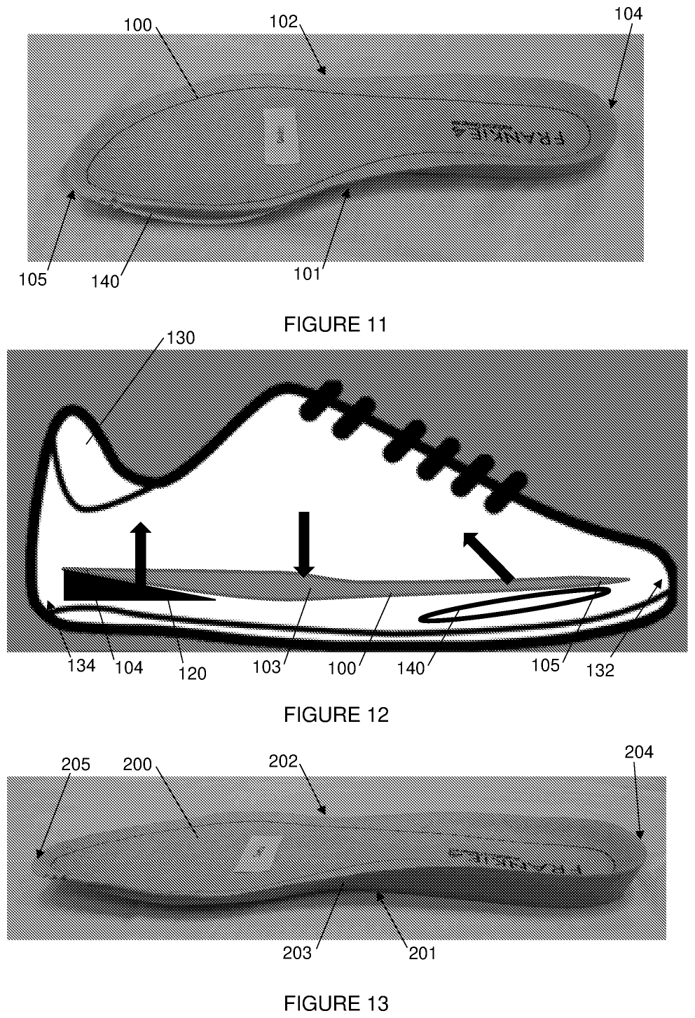

Turning to the forefoot insert 140, it can be seen in FIG. 11 (in use with the contoured full length footbed 100), FIG. 12 (in use with the heel raising insert 120 and forefoot insert in shoe 130) and FIG. 2. The forefoot insert 140 is designed to be used in conjunction with contoured full length footbed 100 and additionally, in some embodiments, the heel raising insert 120. The primary function of the forefoot insert 140 is to provide a better, tighter fit for narrower feet. The forefoot insert 140 can be attached to an underside of contoured full length footbed 100 by any suitable means, including using an adhesive as an example.

By inserting the forefoot insert 140 in a shoe beneath the contoured full length footbed 100 it is possible to provide a tighter fit while maintaining the standard width footbed. The forefoot insert 140, when inserted beneath footbed 100, for example, causes the toe portion 105 of the footbed 100 to be lifted upwardly (toward the top 132 of the shoe 130) to provide the tighter fit. As a result, a variety of different fittings can be provided in the same shoe.

The forefoot insert 140 also provides extra forefoot cushioning in addition to adjusting the fit of the shoe. The width of a shoe is typically indicated as between A-E and is typically based on the width of the foot. Common step sizes are 3/16 of an inch. A particular shoe range can, as an example, be classified as a “C” width fitting but, using the provided forefoot insert 140, can be easily converted to a tighter fitting.

An example of the forefoot insert 140 in use is provided hereafter and in FIG. 12. The embodiment in FIG. 12 is similar to the embodiment shown in FIG. 10 but additionally includes the forefoot insert 140 which is positioned under the contoured full length footbed 100. As noted above, the forefoot insert 140 causes the toe portion 105 of the contoured full length footbed 100 to be deformed or lifted upwardly (as indicated by the upward arrow) to also provide the tighter fitting in addition to the arch support provided by the contoured full length footbed 100 and the heel elevation provided by the heel raising insert 120.

Turning to FIG. 13, there is shown another embodiment of a contoured full length footbed hereinafter referred to as a second, integrally formed contoured full length footbed 200.

The second contoured full length footbed 200 is substantially similar to the contoured full length footbed 100 having a contoured medial side 201 (opposite a lateral side 202) which is configured for a user who has a pronating foot tending toward overpronation or “flat footedness”.

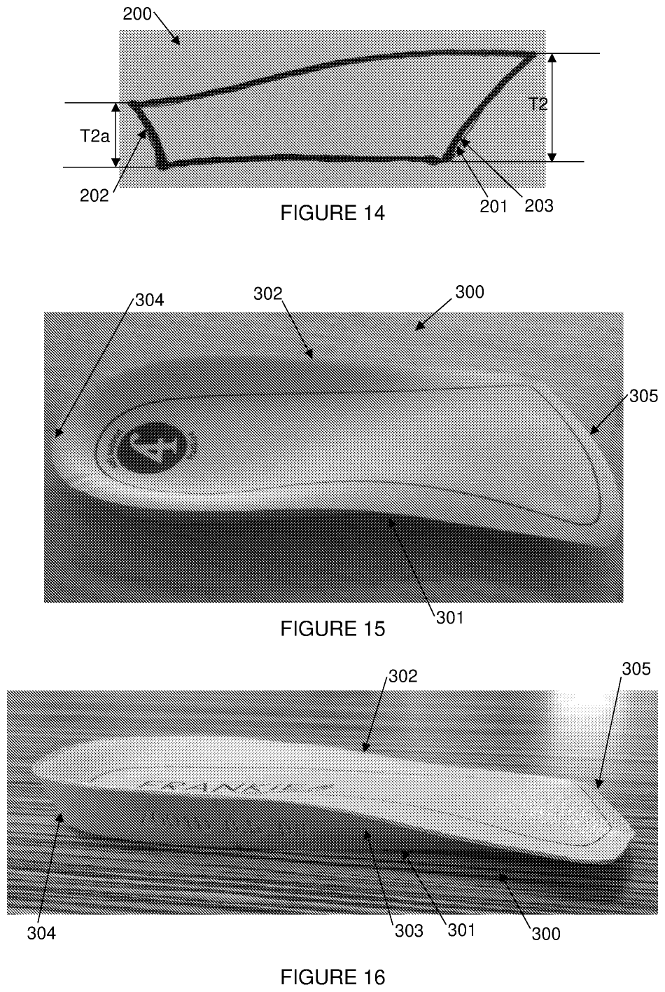

The second contoured full length footbed 200 further includes a contoured arch portion 203 having thickness T2 providing a second degree of arch support (see FIG. 14) located on the medial side 201 of the footbed 200. The contoured arch portion 203 supports the arch and reduces or prevents the foot of a user from excessively pronating to mitigate stress and/or injuries.

The opposing lateral side 202 has a thickness T2a which is less than thickness T2 of the medial side 201.

Thickness T2 of the contoured arch portion 203 of the second contoured full length footbed 200 is less than thickness T1 of the contoured arch portion 103 of the contoured full length footbed 100 and thus provides a lower or reduced degree (comparatively) of cushioning or support. The second contoured full length footbed 200 also includes a heel portion 204 opposite a toe portion 205.

The second contoured full length footbed 200 may also be softer (e.g. have more cushioning or constructed from a softer material) than contoured full length footbed 100.

In some embodiments, a footwear insert assembly is provided having at least one of each of a contoured full length footbed 100 and a second contoured full length footbed 200. Alternatively, a footwear insert assembly having a contoured full length footbed in the form of the second contoured full length footbed 200 and a heel raising insert 120 may be provided.

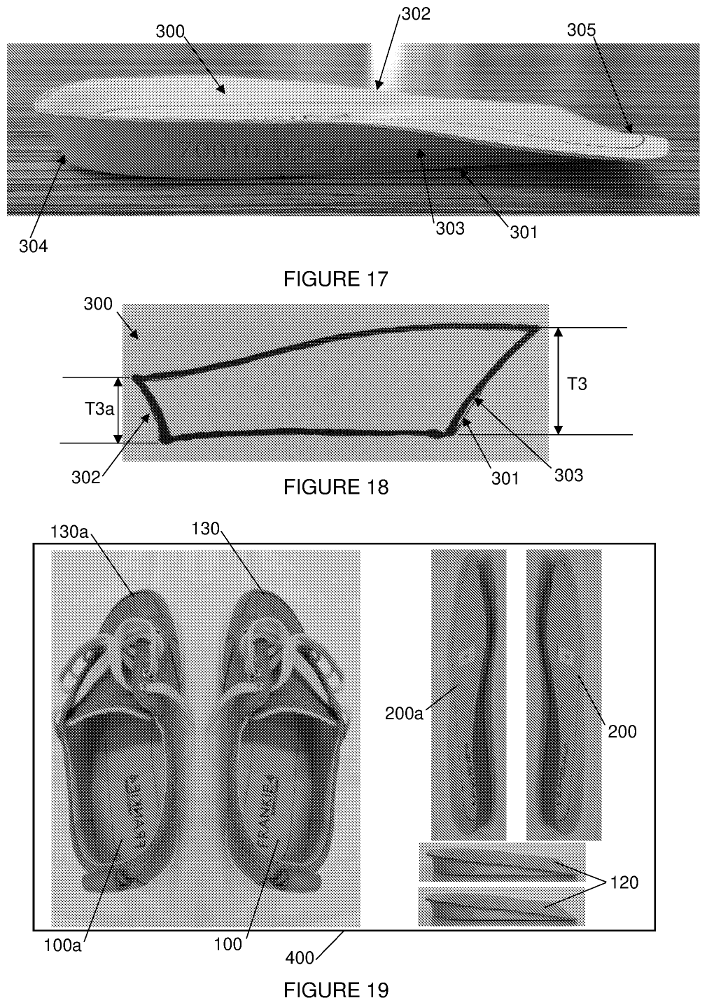

Referring now to FIGS. 15-17, there is shown a contoured half length footbed 300. Similar to the contoured full length footbed 100, the contoured half length footbed 300 includes a contoured medial side 301 (opposite a lateral side 302) and is designed for a user who has a pronating foot tending toward overpronation or “flat footedness”. As with the contoured full length footbed 100, a contoured arch portion 303 having thickness T3 providing a third degree of arch support (see FIG. 18) is located on the medial side 301 of the contoured half length footbed 300. The contoured arch portion 303 supports the arch of the foot and reduces or prevents the foot from excessively pronating to mitigate injuries by supporting the arch of the foot and preventing it from flattening out which is typical during the overpronation motion. Contoured half length footbed 300 also includes a heel portion 304 and a toe-oriented portion 305. Notably the toe-oriented portion 305 does not extend to an end or front portion of a shoe when inserted therein.

Thickness T3 of the contoured arch portion 303 of the medial side 301 may be the same as thickness T1 of contoured arch portion 103 or thickness T2 of contoured arch portion 203, or may be a different thickness to both and thereby provide a different degree of arch support in comparison.

Contoured half length footbed 300 is shown in a left foot configuration but could be easily and readily provided in a right foot configuration.

Turning to FIG. 19, there is shown a container 400 (the container 400 could be a shoebox or any other suitable container) having a pair of shoes (left shoe 130 and right shoe 130a), a pair of contoured full length footbeds 100, 100a (left and right, respectively) and a pair of heel raising inserts 120, which are also left and right and can be inserted into shoes 130, 130a, respectively. Left shoe 130 and right shoe 130a are identical to shoe 130 described herein, differing only in the right or left side configuration. Similarly, contoured full length footbeds 100, 100a are identical to contoured full length footbed 100 described herein, differing only in the right or left side configuration.

In a preferable embodiment shown in FIGS. 21-24, a length of a heel raising insert in the form of heel wedge 600 is approximately half of a length of the contoured full length footbed 100. Thus, in use, the heel wedge 600 extends approximately halfway along the length of the full length footbed 100.

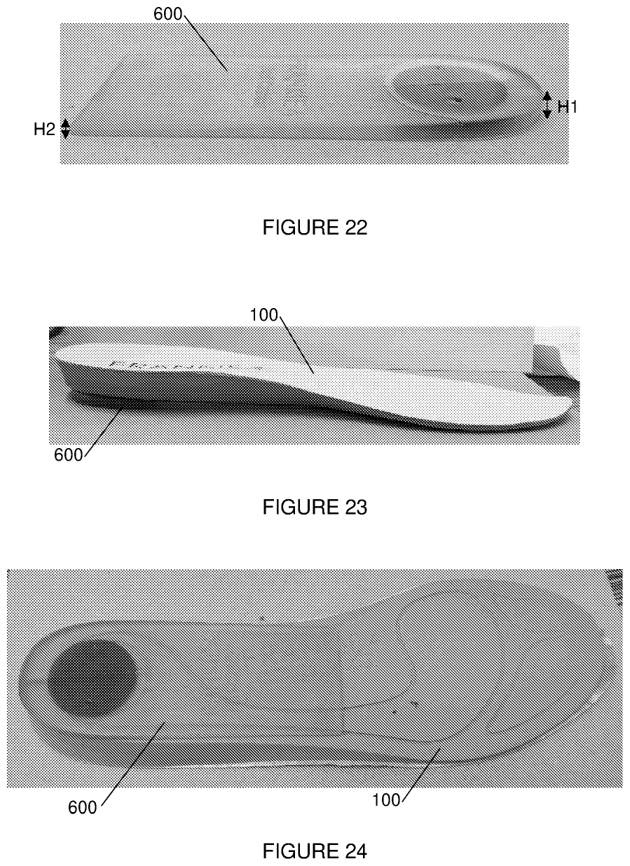

As can be seen in FIG. 22, heel wedge 600 includes a first end 601 (to be located under the heel of the footbed 100) having a first height H1 and a second end 602 (to be located under approximately the middle of the footbed 100) having a second height H2, which is substantially less than H1. In a particularly preferable embodiment, H1 is 5 mm and H2 is 1 mm. The illustrated heel wedge 600 also has a length of approximately 125 mm which equates to a gradient of 4/125 (i.e. 0.032), which is quite small and thus indicates that the slope of the heel wedge 600 is very shallow. Ideally, the gradient of the heel wedge is less than 0.08 and is even more preferably less than 0.05.

In some further embodiments, a heel wedge having a length of approximately 140 mm, a first height of 8 mm and a second height of 1 mm has a gradient of approximately 0.05. In some further embodiments, a heel wedge having a length of approximately 100 mm, a first height of 3 mm and a second height of 1 mm has a gradient of approximately 0.03. Thus, it is preferable that the gradient of the heel wedge be between approximately 0.03 and 0.05.

As will be explained in more detail below, foot pain is very commonly located in the heel or back part of the foot. It is known that a heel raising insert can be effective in reducing pressure on the heel and alleviating foot pain. However, the inventor envisions that a heel raise with a low gradient (i.e. gentle or shallow incline) which extends further along the length of the footbed and therefore further along the underside of the wearer's foot is effective in reducing the stress and pressure on the plantar fascia.

In some embodiments, a pair of forefoot inserts (such as forefoot inserts 140) and/or a pair of half length footbeds (such as contoured half length footbeds 300) and/or a pair of second full length footbeds (such as second full length footbeds 200) could also be provided in the container 400.

The invention allows a shoe to have a range of fitting choices and orthotic support for the customer without requiring the purchase of a different shoe or footbed for each desired fit or foot function.

It is envisioned that, in some embodiments, a pair of shoes in a shoebox will be purchased and the shoebox will contain at least one pair of contoured full length footbeds to be worn for standard width feet which have a pronating foot and a pair of heel inserts to be inserted beneath the contoured full length footbeds. In use, the user would be able to use the contoured full length footbed interchangeably with or without the heel insert (i.e. remove or insert the heel insert).

Furthermore, each shoe can be individually fitted. For example, a customer may pronate with their left foot but not their right foot. In this situation, the customer can insert a first contoured full length footbed having a higher/thicker contoured arch portion in the left shoe. The user can then insert a second contoured full length footbed having a relatively thinner contoured arch portion (compared to the first contoured full length footbed) in the right shoe for the right foot which does not require as much support or correction (compared to the left foot). Thus, the left foot is more supported than the right. Of course, any combination of the inserts/footbeds described throughout the specification can be provided with an individual shoe or pair of shoes. For example, the shoebox may also contain a forefoot insert that can be positioned underneath either of the footbeds in a forward part of the shoe to provide a tighter fit for narrower feet.

In another example, a person may have leg length discrepancies with a left leg that is shorter than the right leg. In this situation, the person may place a heel raising insert in the left shoe and place a contoured full length footbed atop the heel raising insert in the same shoe. In the right shoe, the person simply inserts a contoured full length footbed. The heel raising insert in the left shoe alleviates the discrepancy in length between the left and right legs and allows the person to walk with a normal or regular gait.

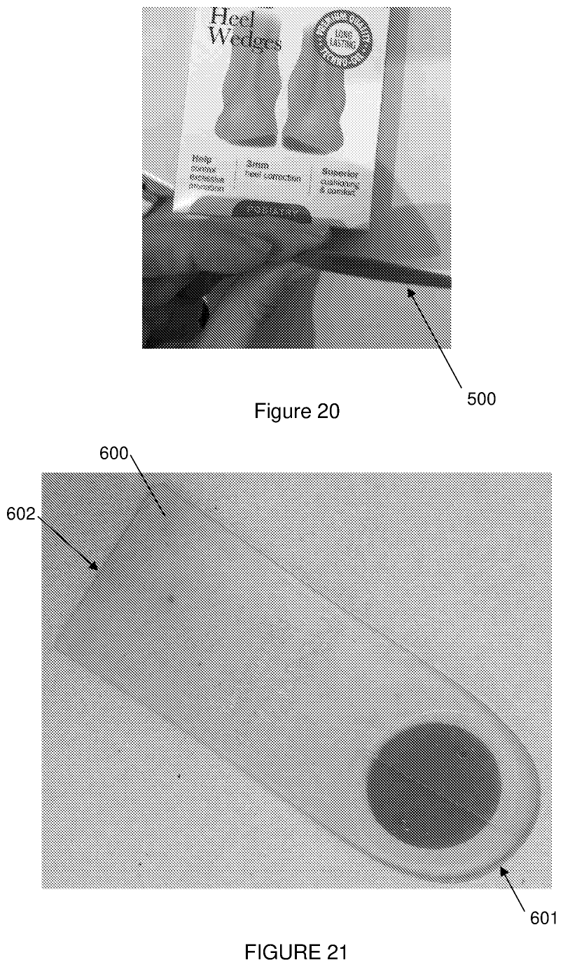

As used herein, the term “heel insert” and the variations thereof refer to an insert that is positioned or located beneath a heel portion of a footbed and atop a heel portion of a shoe. The heel insert may be a heel raising insert that raises or elevates the heel portion of the footbed above the heel portion of the shoe and tilts the heel portion of the footbed in a forward direction away from the rear or back portion of the shoe. The heel insert may, alternatively, be a heel wedge insert having an angled or sloped surface that raises the heel portion of the footbed above the heel portion of the shoe and tilts the heel portion of the footbed in a forward direction away from the rear and toward either the medial side or the lateral side of the footbed or shoe. This tilting of the heel is also commonly known as varus and valgus positions. An example of a heel insert in the form of a varus (i.e. outwardly tilting or laterally tilting) heel wedge 500 is shown in FIG. 20. The heel wedge insert is particularly useful in the management of many painful conditions affecting the plantar area of the heel and midfoot (underneath area) and posterior area of the foot (i.e. behind the heel).

The inserts/footbeds are particularly suitable for use with exercise shoes or running shoes but can be used with any type of shoe. In this regard, the shoe can remain comfortable while also reducing excessive pronation and tension on ligament and tendons while walking or running when necessary.

Aside from the ability to customise a shoe for specific uses, the user can also adjust a shoe based on their own personal preference for the “feel” of a shoe. In particular, a customer may like the look and design of a particular shoe and, using the footwear insert assemblies described herein, can modify the feeling of the fit and foot support of the preferred shoe by interchanging the footbeds as desired to fit their personal preferences and foot support needs.

In addition, stockists or retailers will not need to stock two pairs of the same shoes one pair having only a contoured footbed and the other pair having the contoured footbed paired with a heel insert. Embodiments of the present invention will allow retailers to simply have one shoe design that can be customised to the customer's desires. Advantageously, this minimises the amount of stock stores have to purchase and stock/display.

The term “footbed” used throughout the present specification may comprise insoles, inserts and the like.

As used herein, the term “contoured footbed” and the variations thereof (e.g. full length or half length) refer to a footbed that provides arch support to reduce or eliminate excessive pronation. The contoured footbed provides a supportive feel for a foot, for example by means of a thickening or built up portion of the footbed in a region corresponding to a wearer's arch. The contoured footbed also provides a supportive feeling for a user during wearing and use. A skilled addressee will appreciate that the contoured footbed may also be known as a “posted footbed” or “supportive footbed”. The contoured footbeds as described herein are not flat or neutral footbeds.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step, etc.

The above detailed description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

Throughout the specification and claims (if present), unless the context requires otherwise, the term “substantially” or “about” will be understood to not be limited to the specific value or range qualified by the terms.

1. A footwear insert assembly comprising:
a pair of integrally formed full length footbeds adapted for insertion within an item of footwear, the pair of full length footbeds comprising:
a first contoured footbed having a contoured arch portion, the contoured arch portion of the first contoured footbed having a thickness providing a first degree of arch support;
a second contoured footbed having a contoured arch portion, the contoured arch portion of the second contoured footbed having a thickness that is less than the thickness of the contoured arch portion of the first contoured footbed, the contoured arch portion of the second contoured footbed providing a second degree of arch support;
wherein each of the first contoured footbed and the second contoured footbed is adapted for interchangeable insertion within an item of footwear to thereby produce a supportive feel when worn; and
a heel insert adapted for insertion in the item of footwear beneath one of the full length footbeds of the pair of integrally formed full length footbeds such that the full length footbed and the heel insert cooperate with the footwear to raise a heel portion of the full length footbed, wherein a length of the heel insert is approximately half of a length of the first and second contoured footbeds.

2. The footwear insert assembly of claim 1 further comprising an item of footwear.

3. The footwear insert assembly of claim 1, further comprising at least one of:
a half length footbed adapted for insertion within the item of footwear to provide a wide fitting, wherein the heel insert is insertable beneath the half length footbed; or
a forefoot insert adapted for insertion beneath the full length footbed within the footwear so that the full length footbed and the forefoot insert cooperate with the footwear to produce a narrow fitting.

4. The footwear insert assembly of claim 2, wherein the full length footbed having the heel insert beneath a heel portion of the full length footbed is inserted into the item of footwear to provide a raised heel portion of the full length footbed.

5. The footwear insert assembly of claim 3, wherein the full length footbed having the forefoot insert beneath a forward portion of the footbed is inserted into the item of footwear to provide a narrow fit.

6. The footwear insert assembly of claim 1, wherein each of the first and second contoured footbeds comprises a medial side and a lateral side, and wherein the contoured arch portion of each of the first and second contoured footbeds is located along the respective medial side.

7. The footwear insert assembly of claim 1, wherein the heel insert comprises a heel raising insert adapted to uniformly elevate the heel.

8. The footwear insert assembly of claim 1, wherein the heel insert comprises a heel wedge insert adapted to elevate and tilt the heel in a varus or valgus position.

9. The footwear insert assembly of claim 8, wherein the heel wedge insert comprises a varus wedge insert or a valgus wedge insert.

10. The footwear insert assembly of claim 1, wherein the heel insert comprises a first end having a first height, a second end having a second height and a surface extending between the first end and the second end, wherein the second height is less than the first height, and wherein a gradient of the surface is between approximately 0.02 and 0.08.

11. The footwear insert assembly of claim 10, wherein the gradient of the surface is between approximately 0.03 and 0.05.

12. The footwear insert assembly of claim 10, wherein the gradient of the surface is less than 0.05.

13. A footwear insert assembly comprising:
a container;
a pair of items of footwear located in the container;
two pairs of integrally formed full length footbeds adapted for insertion within an item of footwear of the pair of items of footwear, each pair of integrally formed full length footbeds comprising:
a first contoured footbed having a contoured arch portion, the contoured arch portion of the first contoured footbed having a thickness providing a first degree of arch support;
a second contoured footbed comprising a second contoured footbed having a contoured arch portion, the contoured arch portion of the second contoured footbed having a thickness that is less than the thickness of the contoured arch portion of the first contoured footbed, the contoured arch portion of the second contoured footbed providing a second degree of arch support; and
a pair of heel inserts, each heel insert adapted for insertion beneath one footbed of each of the pairs of footbeds such that the footbed and the heel insert cooperate within the footwear to raise a heel portion of the footbed, wherein a length of each heel insert is approximately half of a length of the first and second contoured footbeds.

14. The footwear insert assembly of claim 13, wherein the container comprises a shoebox.

15. A method for providing a range of at least one of fittings or supports to an item of footwear, the method comprising the steps of:
providing a first contoured full length footbed having a first degree of arch support and a heel insert, wherein the first contoured full length footbed is integrally formed and wherein a length of the heel insert is approximately half of a length of the first contoured footbed;
providing a second contoured full length footbed having a second degree of arch support, wherein the second contoured full length footbed is integrally formed;
inserting the heel insert into an item of footwear at a heel portion of the item of footwear; and
inserting the first contoured full length footbed into the item of footwear, wherein the heel insert is located between a heel portion of the first contoured full length footbed and a heel portion of the item of footwear;
removing the first contoured full length footbed from within the item of footwear; and
inserting the second contoured full length footbed into the item of footwear, wherein the heel insert is located between a heel portion of the second contoured full length footbed and a heel portion of the item of footwear.

16. A footwear insert assembly comprising:
an integrally formed full length footbed adapted for insertion within an item of footwear, the full length footbed comprising a contoured footbed having a contoured arch portion, the contoured arch portion of the contoured footbed having a thickness providing a degree of arch support;
wherein the contoured footbed is adapted for removable insertion within an item of footwear to thereby produce a supportive feel when worn; and
a heel insert adapted for insertion in the item of footwear beneath the contoured footbed such that the contoured footbed and the heel insert cooperate with the footwear to raise a heel portion of the full length footbed, wherein a length of the heel insert is approximately half of a length of the contoured footbed.
